(19) United States
(12) Patent Application Publication
(10) Pub. No.: US 2015/0165172 A1
(43) Pub. Date: Jun. 18, 2015

(54) Wound Drain

(71) Applicant: Billy Russell Wall, II, Arrington, TN (US)

(72) Inventor: Billy Russell Wall, II, Arrington, TN (US)

(21) Appl. No.: 14/491,487

(22) Filed: Sep. 19, 2014

Related U.S. Application Data
(60) Provisional application No. 61/880,049, filed on Sep. 19, 2013.

(51) Int. Cl. A61M 27/00; A61F 13/02

(52) U.S. Cl. CPC A61M 27/00; A61F 13/0216; A61F 13/0266

(57) ABSTRACT

The wound drain has an integral pliable base member with an adhesive backing that affixes the drain to the patient's skin and holds it in place so that the drain line can be fitted into the wound cavity. The base member is shaped and configured to affix to the skin adjacent the wound cavity and has a domed top, tapered sides or “wings” and a substantially flat bottom surface. The pliable base member stretches and contorts so that it lies flat against the patient's skin and conforms around and into body creases and folds. The bottom of the base member is coated with an adhesive, which is covered by a removable non-stick backing.

This application claims the benefit of U.S. Provisional Application No. 61/880,049 filed Sep. 19, 2013, the disclosure of which is hereby incorporated by reference.

This invention relates a surgical “wound” drain for wound therapy and in particular a drain having an integrated body for stabilizing the drain line within the wound cavity.

### BACKGROUND AND SUMMARY OF THE INVENTION

Surgical wound drains, such as the “Jackson-Pratt” or “JP” drain, are commonly used as a post-operative drain for collecting bodily fluids from surgical sites. Wound drains generally consist of a drain line, which is a perforated sterile tube that is placed directly within the wound cavity. The drain line is often connected to a fluid collection apparatus and/or vacuum source, such as wall suction or a simple suction bulb via a fluid line, which is simply a length of flexible tubing.

The drain line is typically cut to length to rest within the wound with a fluid line extending out from the wound. An occlusive dressing is applied over the wound cavity and drain line to create a hermetic seal. Traditionally, occlusive dressings are difficult to work with and manipulate, but are critical for providing the hermetic seal around a wound for negative pressure wound therapy. Often patient movement can dislodge or displace the drain line under the occlusive dressing causing a vacuum leak. Creating a sufficient seal is also complicated by the contours, creases and folds in the patient's skin around the wound. Heretofore, gaskets and sealants have been used around drain lines and occlusive dressings to maintain the hermetic seals around wound cavities.

The present invention seeks to provide a surgical wound drain apparatus that simplifies and improves patient care by freeing the care giver from having to hold a drain line in place within a wound as an occlusive dressing is applied over the wound. The wound drains of this invention in various embodiments are intended for use in combination with a conventional occlusive dressing and certain embodiments can be used simultaneously with both negative and positive pressure wound therapy protocols. In each embodiment, the wound drain has an integral pliable base member with an adhesive backing that affixes the drain to the patient's skin and holds it in place so that the drain line can be fitted into the wound cavity. The base member is shaped and configured to affix to the skin adjacent the wound cavity and has a domed top, tapered sides or “wings” and a substantially flat bottom surface. The pliable base member stretches and contorts so that it lies flat against the patient's skin and conforms around and into body creases and folds. The bottom of the base member is coated with an adhesive, which is covered by a removable non-stick backing. When the backing is removed, the base member can be affixed directly to the skin adjacent a wound for conveniently positioning and securing the drain line within the wound, while an occlusive dressing is applied over the wound and drain.

The wound drains of this invention simplify and improve patient care by freeing the care giver from having to hold a drain line in place within a wound as an occlusive dressing is applied over the wound. The adhesive coating on the base member allows the drain apparatus to be affixed directly to the patient's skin adjacent the wound cavity to position and hold the drain line in place within the wound cavity as the occlusive dressing and drain cover are applied. With the drain line held in place within the wound cavity, the care provider does not have to manually hold the drain in place and is free to use both hands to apply the occlusive dressings and drain cover. The shape and contour of the base member provide a large smooth surface area that the occlusive dressings adhere to for creating a hermetic seal around the drain part, as well as the wound cavity. The pliable body of the base member allows it to conform to the contours and creases of the skin around a wound cavity, while still maintaining a hermetic seal with the skin and prevents the drain line from moving or unseating as the patient moves, which may create a leak in the hermetic seal. The use of adhesives on the bottom surface of the base member eliminates the need for gaskets and sealants to be applied around fluid and drain lines in order to maintain a hermetic seal around the drain and the wound cavity.

The above described features and advantages, as well as others, will become more readily apparent to those of ordinary skill in the art by reference to the following detailed description and accompanying drawings.

### BRIEF DESCRIPTION OF THE DRAWINGS

The present invention may take form in various system and method components and arrangement of system and method components. The drawings are only for purposes of illustrating exemplary embodiments and are not to be construed as limiting the invention.

The drawings illustrate the present invention, in which:

FIG. 1 is a top view of an embodiment of the wound drain with its adhesive cover;

FIG. 2 is a perspective view of the wound drain of FIG. 1

FIG. 3 is a perspective view of the adhesive covering of FIG. 1;

FIG. 4 is a partial perspective view of the end of the dual lumen connection line, which forms part of the sound drain of FIG. 1;

FIG. 5 is a partial perspective view of the end of the dual lumen drain line, which forms part of the wound drain of FIG. 1;

FIG. 6 is a perspective view of the wound drain being positioned adjacent a wound cavity with the adhesive cover and the occlusive dressing positioned over a wound site;

FIG. 7 is a perspective view of the wound drain secured to the body adjacent a wound cavity with the drain line seated within the wound cavity and covered by the occlusive dressing;

FIG. 8 is a perspective view of the adhesive cover positioned over the wound drain and the occlusive dressing to hermetically seal the drain line within the wound cavity;

FIG. 9 is a top view of a second embodiment of the wound drain of this invention;

FIG. 10 is a partial perspective view of the end of an alternative single lumen connection line used in the wound drain of FIG. 9;

FIG. 11 is a partial perspective view of the end of an alternative drain line used in the wound drain of FIG. 9;

FIG. 12 is a perspective view of a second embodiment of the wound drain of this invention;

FIG. 13 is a top view of the wound drain of FIG. 12;

FIG. 14 is a perspective view of the wound drain of FIG. 11 being positioned over a wound cavity on patient's leg;

FIG. 15 is a perspective view of the wound drain of FIG. 11 secured to the body adjacent a wound cavity with the drain line seated within the wound cavity;

FIG. 16 is a perspective view of the occlusive dressing positioned over the wound drain of FIG. 11; and

FIG. 17 is a perspective view of the occlusive dressing covering the wound site and the wound drain of FIG. 11.

### DESCRIPTION OF THE PREFERRED EMBODIMENT

In the following detailed description of the preferred embodiments, reference is made to the accompanying drawings that form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical, structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

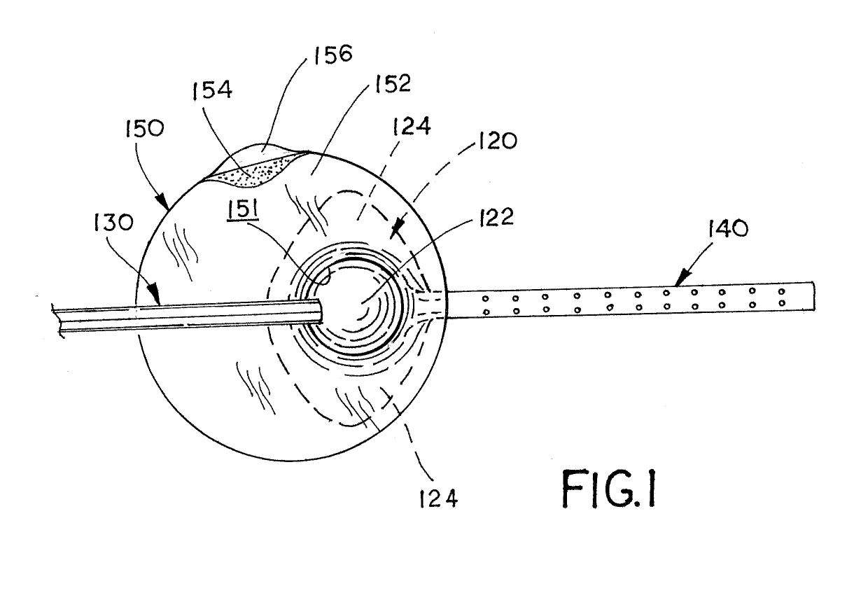

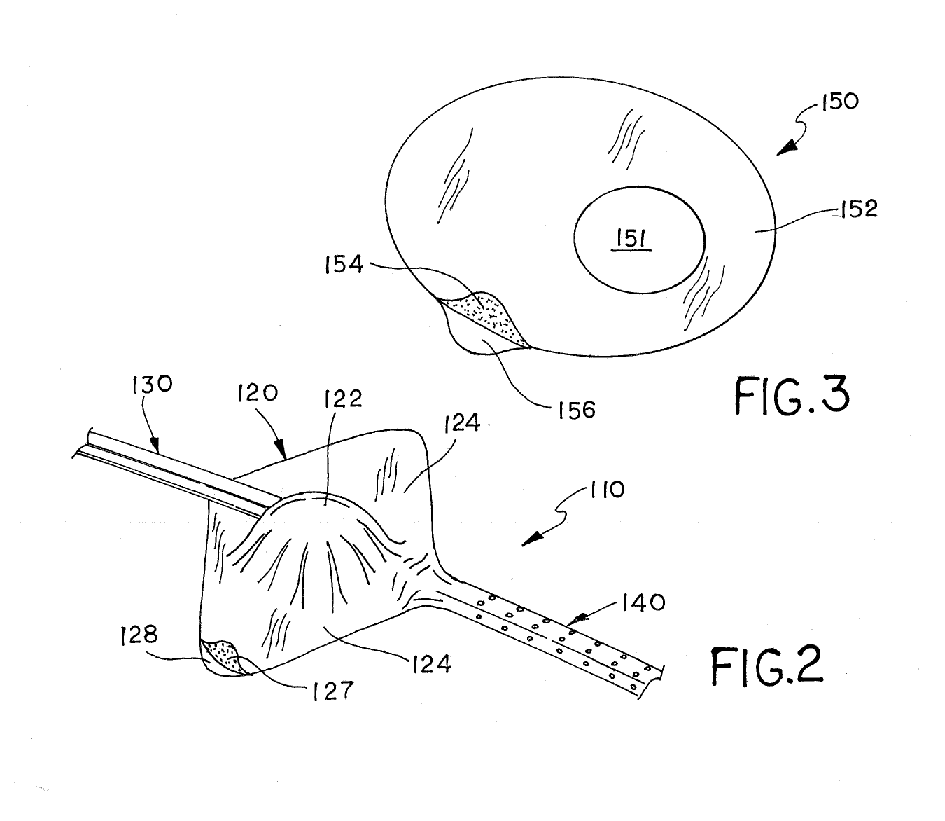

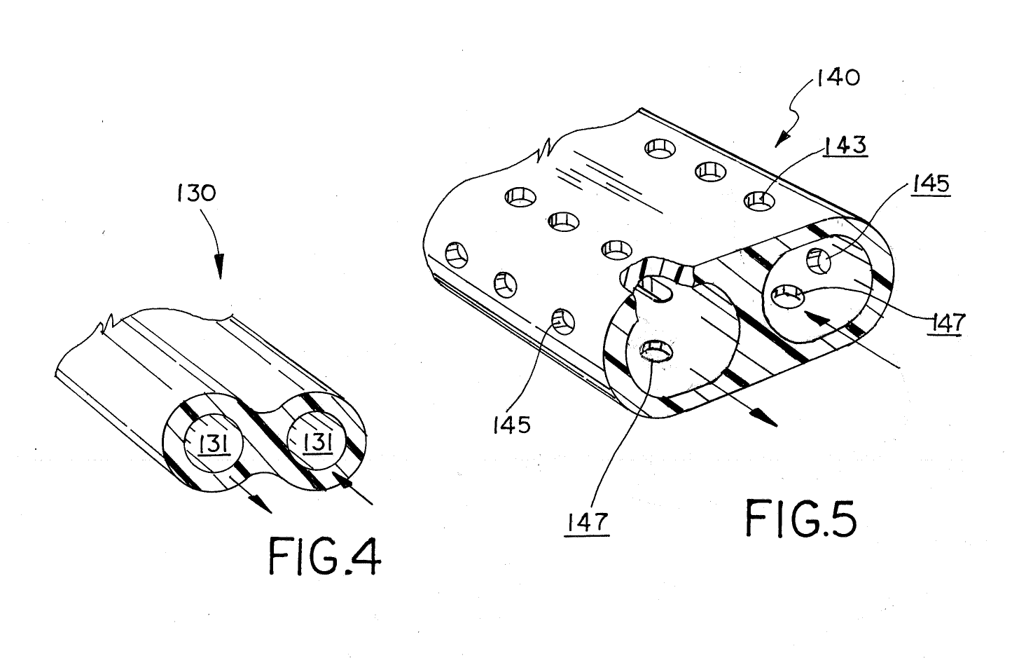

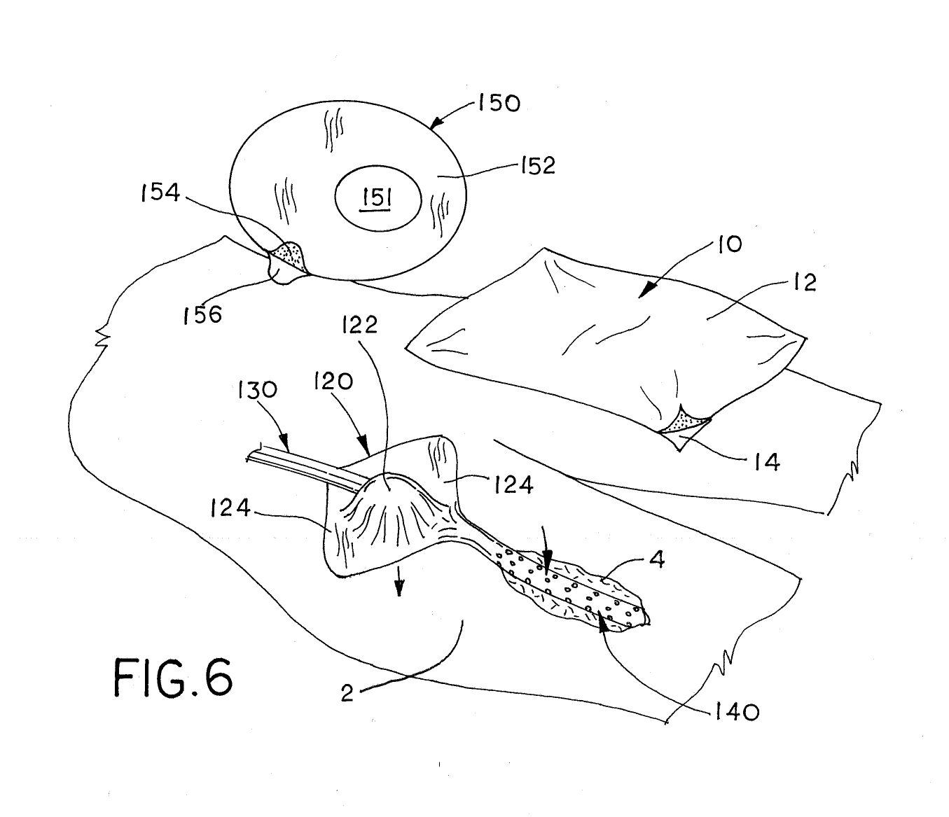

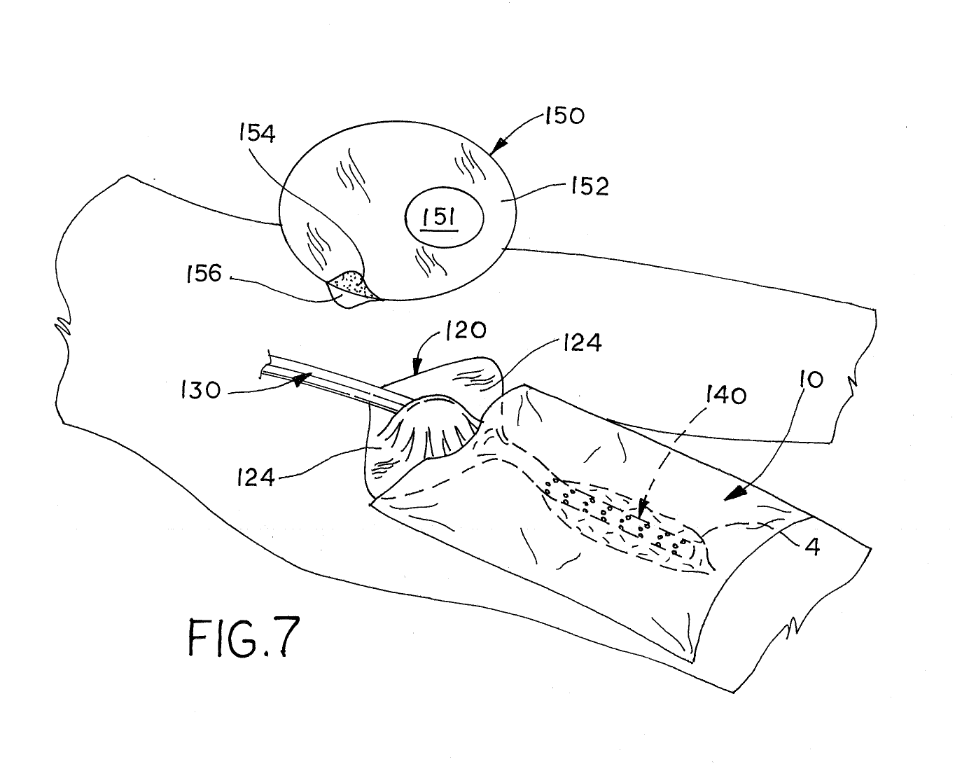

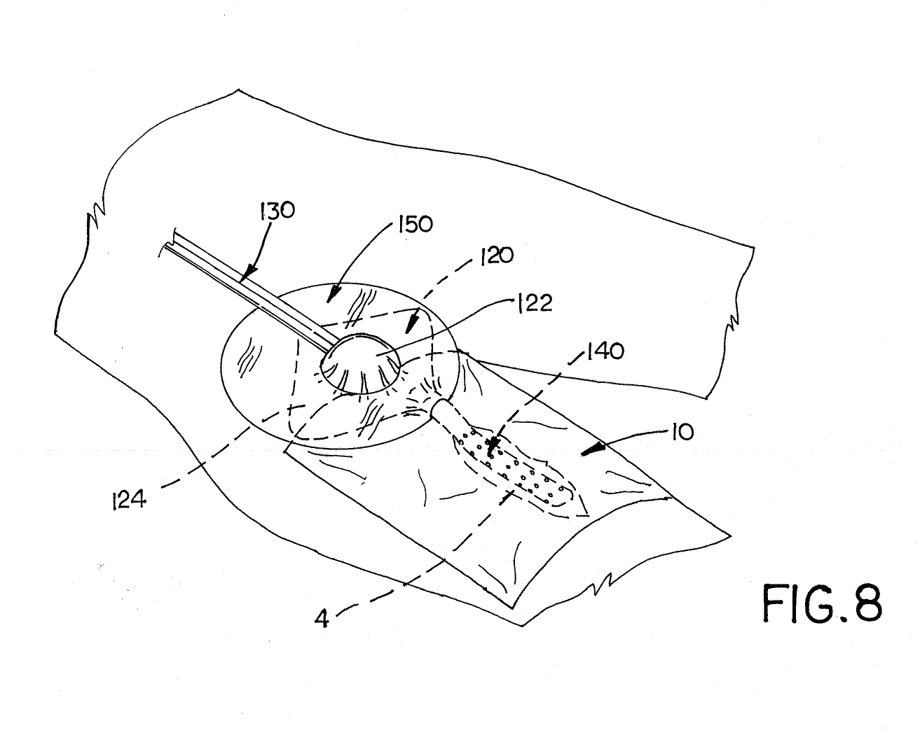

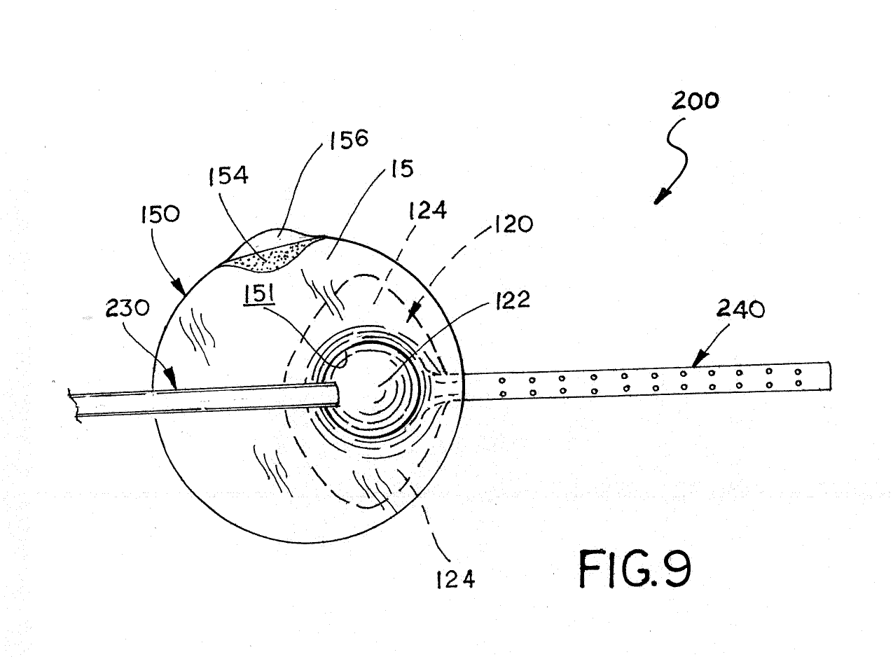

Referring now to the drawings, FIGS. 1-8 illustrate an embodiment of the wound drain of this invention, which is designated generally as reference numeral 100. As shown in FIGS. 6-8, wound drain 100 is intended for use in combination with a conventional occlusive dressing 10. Occlusive dressing 10 is an air- and water-tight trauma dressing commonly used in surgical and wound therapies. Occlusive dressing 10 generally consists of a thin polymer film 12, an adhesive coating 13 and a removable non-stick backing 14 covering the adhesive sides of the film. As shown, wound drain 100 has a two piece design consisting of a pliable drain part 110 and a separate annular adhesive drain cover 150, which covers the drain part and provides a secure hermetic seal around the drain part and the wound cavity in conjunction with occlusive dressing 10 (FIG. 8). Occlusive dressing 10 is dimensioned and sized to cover the wound cavity and at least a portion of drain part 110. Drain cover 150 is designed and intended to cover and seal around drain part 110. Drain cover 150 is a conventional occlusive dressing having a polymer film 152 coated with an adhesive 154 and covered by a removable non-stick backing 156. Drain cover 150 is cut into an annular shape with an offset opening 151, which is shaped to receive the domed top 122 of base member 120, which helps position and align the cover relative to drain part 110.

Drain part 110 is molded or otherwise formed from a silicone rubber or similar material suitable for surgical and medical use. Drain part 110 has a base member 120 and integral connection line 130 and a perforated drain line 140. The body of base member 120 has a domed top 122 with a substantially flat bottom surface 126 and flat tapered peripheral flanges or side “wings” 124 . Bottom surface 126 and side wings 124 are shaped and contoured to overlie and affix to the patient's skin adjacent the wound cavity 2. As shown, side wings 124 of base member 120 taper laterally outward from domed top 122 to provide a large surface area for adhering the base member to the skin adjacent a wound cavity. Base member 120 and particularly side wings 124 are pliable so that the base member can be contorted to conform to the patient's body so that the base member lies flat against the patient's skin even over, around and into body creases and folds. The flat bottom surface 126 of base member 120 is coated with an adhesive coating 127, which is covered by a removable non-stick backing 128. When backing 128 is removed, base member 120 can be affixed to the skin around a wound cavity and held in place by the adhesive coating 127. Adhesive coating 127 and removable backing 128 used on base member 120 are of the type readily known in the medical field and therefore not described in detail herein. It should be noted however, that the adhesive is selected to affix the base member to the body adjacent the wound cavity 2 without adversely affecting the skin around the wound cavity or the wound treatment therapy.

Connection line 130 and perforated drain line 140 are lengths of pliable silicone tubing. Connection line 130 extends integrally from a mid point in domed top 122 of base member 120. Drain line 140 is designed to be embedded directly into a wound cavity. Drain line 140 integrally extends from the rear of base member 120 diverging downward out of domed top 122 with the bottom of drain line 140 being continuous with the bottom surface 126 of the base member.

Wound drain 100 is configured for use in combined negative and positive pressure wound therapy applications. As such, connection line 130 and drain line 140 are configured as dual lumen tubing (two separate flow channels) integrally connected to and in corresponding open fluid communication through the body of base member 120. Connection line 130 is configured to connect to the pump and fluid collection apparatus (not shown) so that one lumen is in communication with the positive air/fluid pressure port of the pump/fluid collection apparatus and the other lumen is in communication with the negative air/fluid pressure port of the apparatus. In addition, the use of a dual lumen fluid line allows for fluid delivery to the wound cavity, as well as fluid drainage from the wound site. It should be noted that in a negative pressure only wound therapy application, both lumens of the connection line may be connected to the negative pressure port of the pump/fluid collection apparatus. In such case, the use of a dual lumen connection line helps reduce the opportunity for line clogs and fluid flow disruption. Drain line 140 has a plurality of spaced openings 143, 145 and 147, which allow air/fluid communication into and out of drain part 110. Each lumen of drain line 140 has its own row of spaced slots 147 formed in the bottom of drain line 140 and a row of spaced drain holes 143 and 145 formed in the top and sides of drain line 140, respectively.

In use, the wound site is cleaned and prepared before wound drain 100 and occlusive dressing 10 are applied. Once cleaned and prepared, drain part 110 and drain cover 150 are removed from their packaging (not shown) along with the desired occlusive dressing 10. Next, base member 120 is affixed to the patient's skin adjacent wound cavity 4. Adhesive backing 128 is manually peeled from base member 120 exposing adhesive coating 127, which adheres the base member to the skin. Base member 120 lies flat against the patient's skin with wings 124 spread laterally following the curves and contours of the skin adjacent wound cavity 4. Once base member 120 is affixed to the patient's skin, drain line 140 may be cut to length and inserted into wound cavity 4. With base member 120 affixed to the patient's skin and drain line 140 seated within the wound cavity, occlusive dressing 10 is applied to cover the wound cavity and at least partially cover the base member. The adhesive back 16 is peeled off to expose the adhesive coating 14, which adheres occlusive dressing 10 to the skin around wound cavity 4 and base member 120. Occlusive dressing 10 is applied ideally to lay flat over the skin and provide a hermetic seal around wound cavity 4. Lastly, drain cover 150 is applied over drain member 120. As shown, fluid line 130 passes through opening 151 so that drain cover 150 overlies base member 120 with fluid line 130 exposed atop the drain cover. Again, adhesive backing 156 is peeled off to expose adhesive coating 154, which adheres drain cover 150 to base member 120, a portion of occlusive dressing 10 and the skin around the base member. It should be noted that occlusive dressing 10 is partially sandwiched between base member 120 and drain cover 150.

FIGS. 9-11 illustrate a second embodiment of the wound drain of this invention, which is designated generally as reference numeral 200. Wound drain 200 is identical in design and function as wound drain 100, except that connection line 230 and drain line 240 are configured as single lumen tubing. As shown, connection line 230 is a conventional single lumen silicone tubing, but drain line 240 has a generally oval or rectangular cross section with smooth rounded edges defining a single internal drain channel 241. An integral top support rib 242 extends downward longitudinally into drain channel 241 from the top of the drain line 240. Two integral bottom support ribs 246 extend upward longitudinally into drain channel 241 from the bottom of drain line 240. Top and bottom support ribs 242 and 246 provide structural integrity to drain line 240, which prevents drain channel 241 from collapsing under any negative pressure used to draw fluids from the wound cavity. Again, drain line 240 has a plurality of openings 243, 245 and 247 through which fluid enters drain line 240.

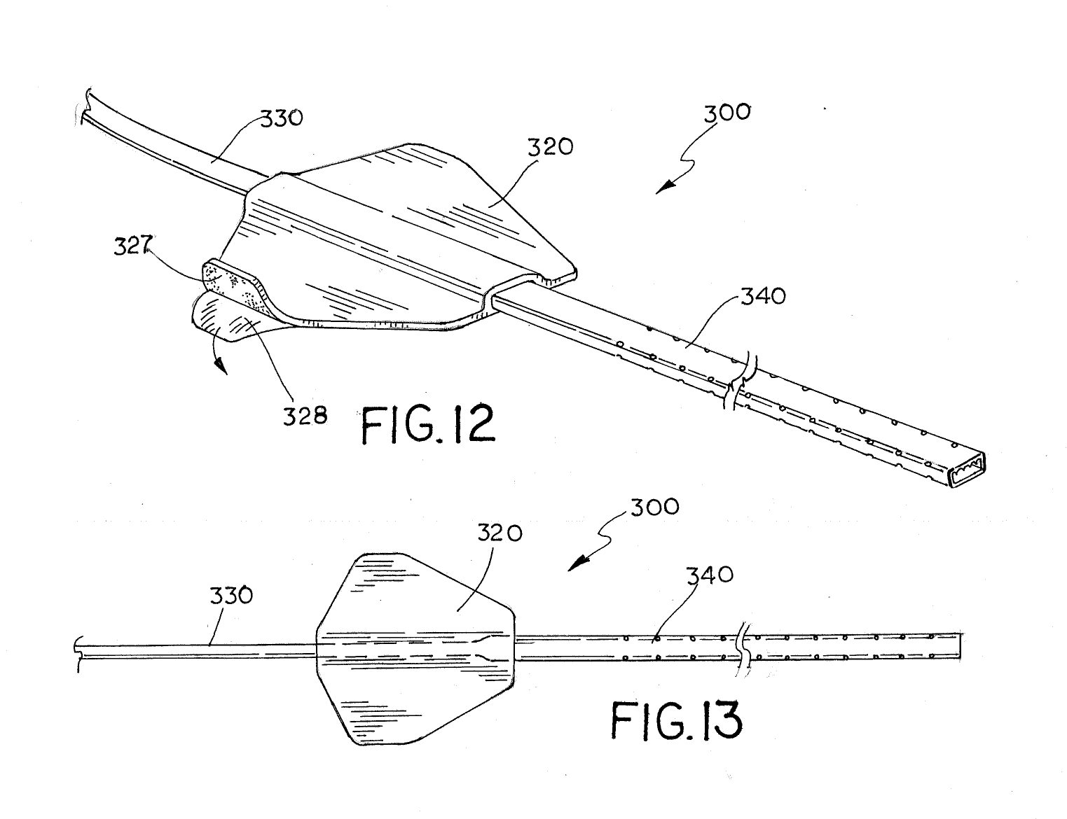

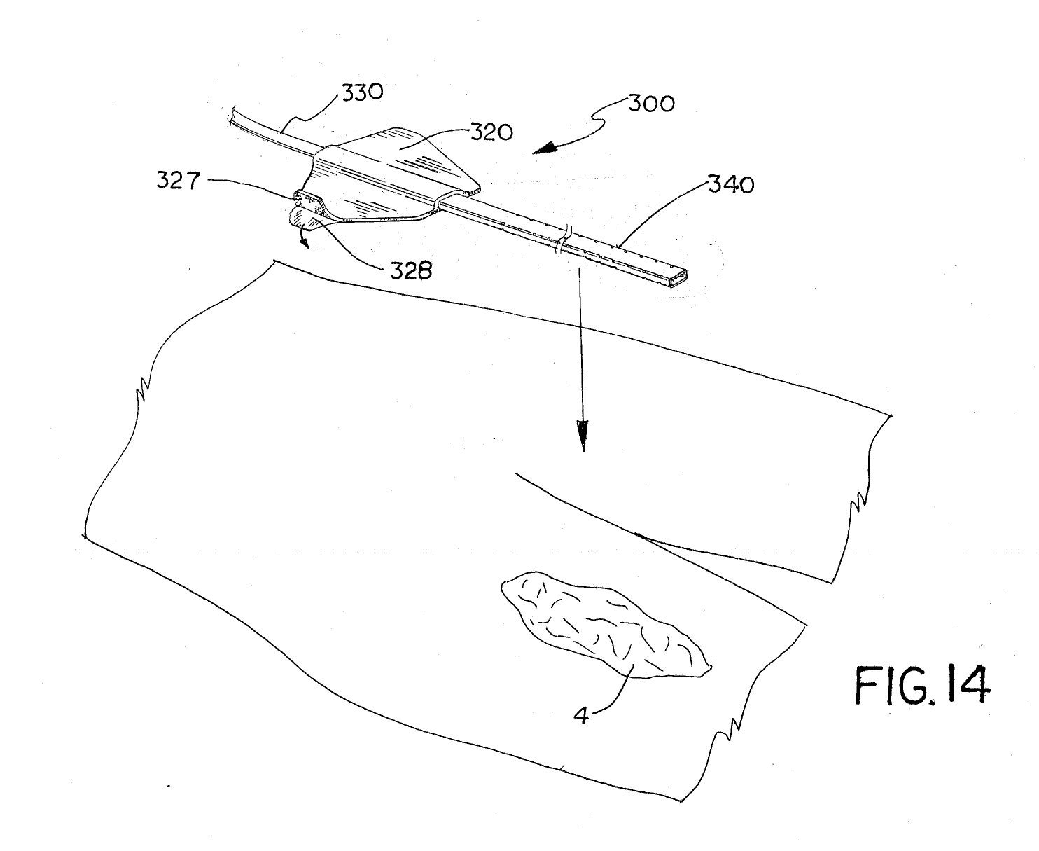

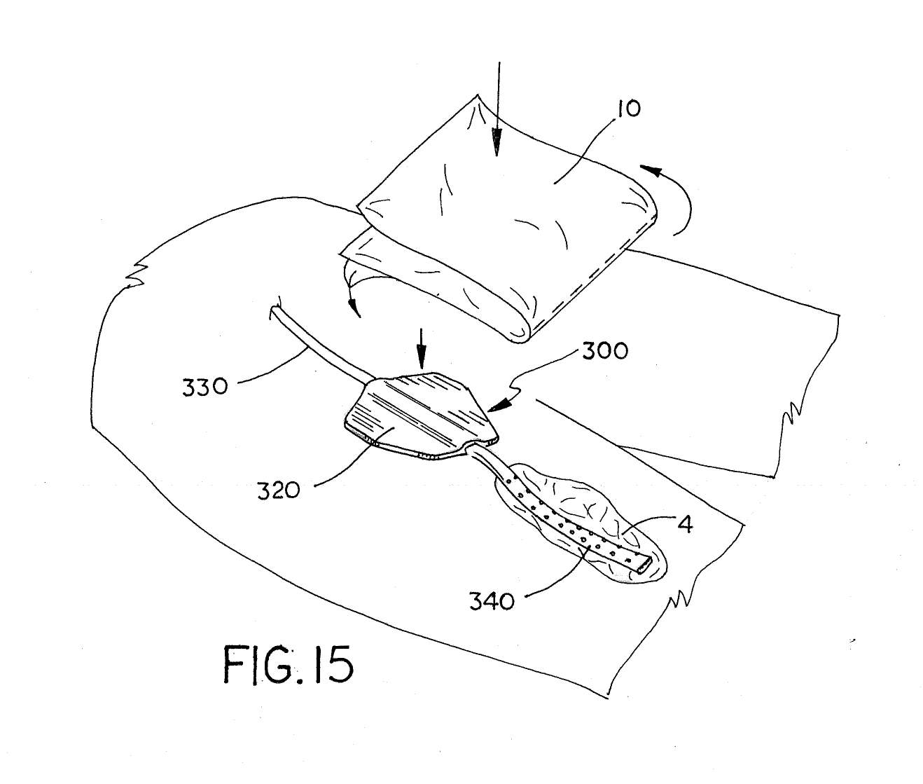

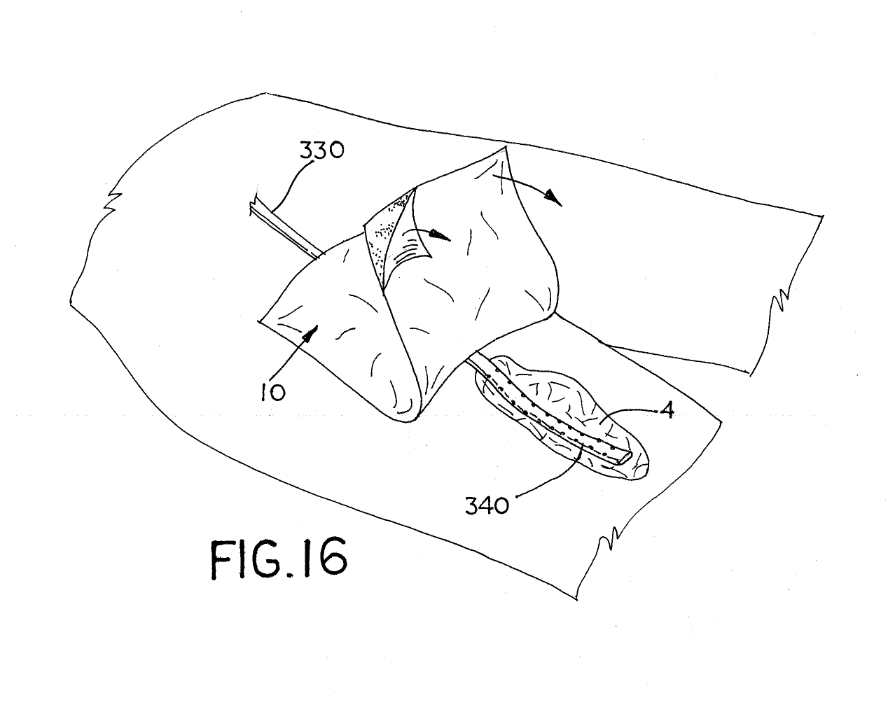

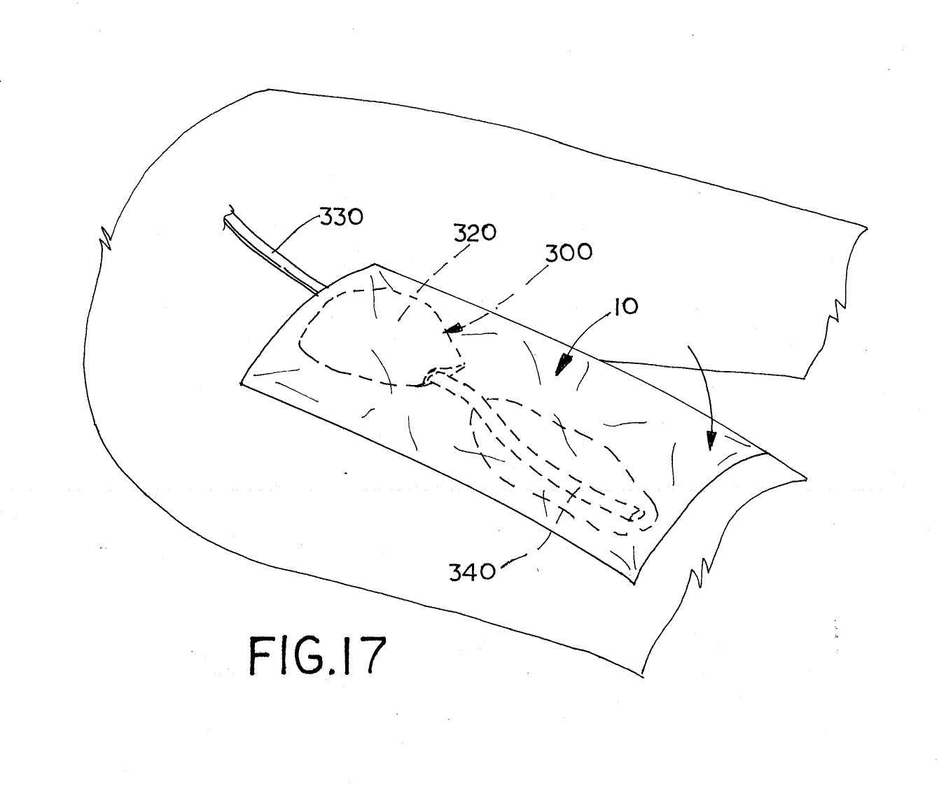

FIGS. 12-17 illustrate another third embodiment of the wound drain of this invention, which is designated generally as reference numeral 300. Wound drain 300 is intended for use in combination with a conventional occlusive dressing 10, but uses a single piece design consisting of a pliable silicone drain part without the additional adhesive covering of wound drain 100.

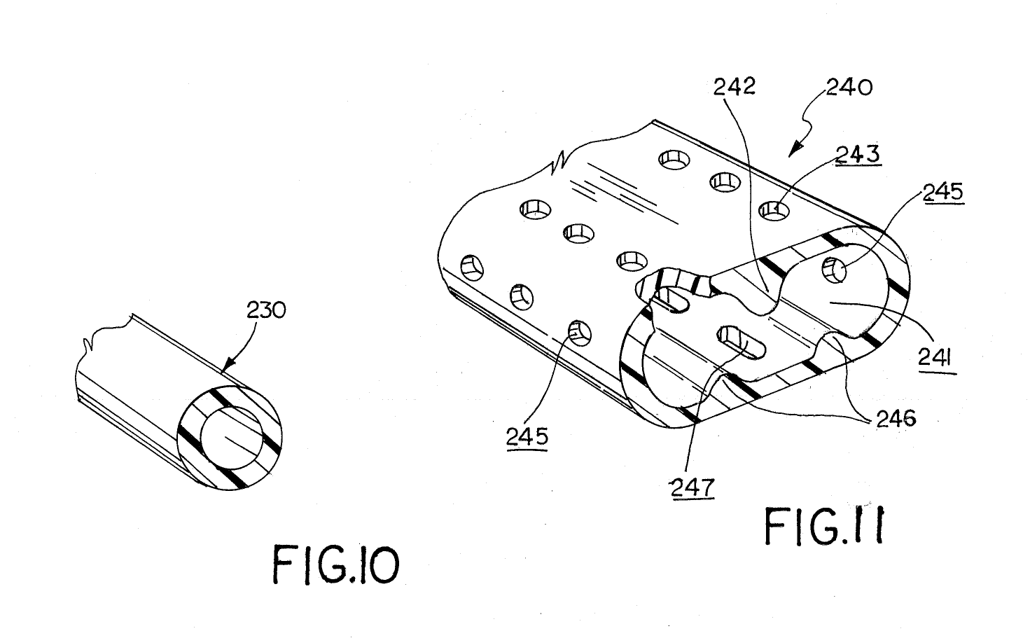

As shown, wound drain 300 is configured as a single lumen drain, but may be configured as a dual lumen drain as desired. Wound drain 300 is molded or otherwise formed from a silicone rubber or similar material and has a flat base member 320 and integral connection line 330 and a perforated drain line 340. As shown, connection line 330 and drain line 340 are formed as single lumen tubing similar in construction and function as connection line 230 and drain line 240 of FIGS. 10 and 11. The integral body of base member 320 is generally flat and like drain part 110, is configured to have pliable peripheral flanges or wings that provide a large surface area that is affixed to the skin adjacent the wound cavity 2. Base member 320 is pliable so that it can be contorted to conform to the patient's body so that the base member lies flat against the patient's skin even over, around and into body creases and folds. As with the above discussed embodiment, the flat bottom surface of base member 320 is coated with an adhesive coating 327, which is covered by a removable non-stick backing 328. When backing 328 is removed, base member 320 can be affixed to the skin around a wound cavity and held in place by the adhesive coating 327.

Wound drain 300 is applied and used in substantially the same manner as wound drain 100. Once the wound site is cleaned and prepared, wound drain 300 is affixed to the patient's skin adjacent wound cavity 4. Adhesive backing 328 is manually peeled from base member 320 exposing adhesive coating 327, which adheres the base member to the skin. Base member 320 lies flat against the patient's skin following the curves and contours of the skin adjacent wound cavity 4. Once base member 320 is affixed to the patient's skin, drain line 340 may be cut to length and inserted into wound cavity 4. With wound drain 300 in place, occlusive dressing 10 is applied to cover both wound cavity 4 and wound drain 300, which provides a hermetic seal around wound cavity 4.

One skilled in the art will note several advantages to the wound drain of this invention. The shaped and contour of the base member provide a large smooth surface area that the occlusive dressings adhere to for creating a hermetic seal around the drain part, as well as the wound cavity. The pliable body of the base member allows it to conform to the contours and creases of the skin around a wound cavity, while still maintaining a hermetic seal with the skin and prevents the drain line from moving or unseating as the patient moves, which may create a leak in the hermetic seal. The use of adhesives on the bottom surface of the base member eliminates the need for gaskets and sealants to be applied around fluid and drain lines in order to maintain a hermetic seal around the drain and the wound cavity.

The wound drain apparatus of this invention simplifies and improves patient care by freeing the care giver from having to hold a drain line in place within a wound as an occlusive dressing is applied over the wound. The adhesive coating on the base member allows the drain apparatus to be affixed directly to the patient's skin adjacent the wound cavity to position and hold the drain line in place within the wound cavity as the occlusive dressing and drain cover are applied. With the drain line held in place within the wound cavity, the care provider does not have to manually hold the drain in place and is free to use both hands to apply the occlusive dressings and drain cover.

It should be apparent from the foregoing that an invention having significant advantages has been provided. While the invention is shown in only a few of its forms, it is not just limited but is susceptible to various changes and modifications without departing from the spirit thereof. The embodiment of the present invention herein described and illustrated is not intended to be exhaustive or to limit the invention to the precise form disclosed. It is presented to explain the invention so that others skilled in the art might utilize its teachings. The embodiment of the present invention may be modified within the scope of the following claims.

I claim:

1. A wound drain adapted for uses with an occlusive dressing to communicate fluids form and/or to a wound cavity, the wound drain comprising:
a pliable base member adapted to overlie and affix to a patient's skin adjacent a wound cavity, the base member has a flat bottom face and an adhesive applied to the bottom face for affixing the base member to the patient's skin;
an elongated connection line integrally extending from the base member and adapted for connection to a wound therapy apparatus for either drawing fluid from the wound cavity or delivering fluid to the wound cavity; and
an elongated integral drain line integrally extending from the base member in open fluid communication with the connection line through the base member and adapted for insertion within the wound cavity.

2. The wound drain of claim 1 wherein the drain line has a rectangular cross section, and a top, bottom and opposed sides defining a tubular interior.

3. The wound drain of claim 2 wherein the drain line has a first plurality of openings formed in each of the opposed drain line sidewalls, a second plurality of openings formed in the drain line top and a third plurality of openings formed in the drain line bottom.

4. The wound drain of claim 2 wherein the drain line has a longitudinal rib extending into the tubular interior from the top.

5. The wound drain of claim 2 wherein the drain line has a pair of parallel longitudinal ribs extending upward into the tubular interior from the bottom.

6. The combination of claim 1 wherein the base member has a domed top surface.

7. The wound drain of claim 1 and a drain covering adapted to overlie the base member and partially cover the occlusive dressing when applied over the wound cavity to securely adhere to each of the occlusive dressing, the base member and the skin around the wound cavity for hermetically sealing the drain line within the wound cavity.

8. The wound drain of claim 1 wherein the base member has flat pliable peripheral flanges shaped and adapted to contort and conform over and to the patient's skin.

9. The wound drain of claim 1 and a removable film covering the adhesive on the bottom face of the base member.
